(19) United States
(12) Patent Application Publication
(10) Pub. No.: US 2016/0060351 A1
(43) Pub. Date: Mar. 3, 2016

(54) Use of C5AR Antagonists

(71) Applicant: NOVO NORDISK A/S, Bagsvaerd (DK)

(72) Inventors: Ellen Christina Andersson, Virum (DK); Anneline Nansen, Broenshoej (DK); Pernille Autzen Usher, Vanloese (DK)

(73) Assignee: NOVO NORDISK A/S, Bagsvaerd (DK)

(21) Appl. No.: 14/888,141

(22) PCT Filed: May 8, 2014
(86) PCT No.: PCT/EP2014/059477

§ 371 Date: Oct. 30, 2015

Related U.S. Application Data
(60) Provisional application No. 61/821,336, filed on May 9, 2013.

(30) Foreign Application Priority Data
May 8, 2013 (EP) 13166973.1
Jan. 21, 2014 (EP) 14151982.7

(51) Int. Cl. C07K 16/28

(52) U.S. Cl. CPC C07K 16/2896; C07K 2317/76; A61K 2039/505

(57) ABSTRACT

C5a and C5aR stimulate the inflammatory response and C5aR antagonist can counteract this function performing as an anti-inflammatory agents. This functionality is considered useful in treatment of inflammatory diseases including auto immune diseases. In addition it has been found that C5a and C5aR have a function in relation to bone homeostasis and remodelling and that C5aR antagonists are useful for treatment of bone damage, such as bone erosion and loss of bone density and for treatment of bone damage and cartilage damage.

### TECHNICAL FIELD

The present invention concerns additional therapeutic uses of C5aR antagonists in particular in relation to bone damage and diseases and disorders related thereto.

### BACKGROUND

C5aR is the receptor for the A fragment of complement factor 5 (C5a). The receptor is expressed on a number of different cell types including leukocytes. C5aR belongs to the family of seven transmembrane G-protein-coupled receptors and is a high affinity receptor for C5a, with a Kd of ˜1 nM. The structure of the receptor conforms to the seven transmembrane receptor family, with an extracellular N-terminus being followed by seven transmembrane helices connected by interhelical domains alternating as intracellular and extracellular loops, and ending with an intracellular C-terminal domain.

Inhibition of the C5a responses with C5aR antagonists reduces the acute inflammatory response mediated via C5a without affecting other complement components. To this end, C5aR peptide antagonists and anti-C5a receptor antibodies have been previously described (Watanabe et al., 1995 Journal of Immunological Methods 185: 19-29; Pellas et al., 1998; Konteatis et al., 1994; Kaneko et al., 1995; Morgan et al., 1993). For example, WO 95/00164 describes antibodies directed against an N-terminal peptide (residues 9-29) of C5aR. WO 03/062278 described antibodies reacting with the 2^{nd} extracelluar loop (including clones referred to as 7F3, 6C12 and 12D4). Further C5aR antibodies have been described in WO/022390 and recently WO2012/168199. All data confirming that effective blocking of C5a binding to its receptor C5aR, can inhibit C5a stimulated migration of neutrophils in vitro, and preventing inflammation in animal models.

C5a is a pro-inflammatory protein that has been implicated in inflammatory diseases such as in the pathogenesis of rheumatoid arthritis and the relevance of C5a and C5aR in rheumatoid arthritis has been confirmed in mice genetically deficient in C5aR, which are less prone to the induction of arthritis (Ji et al. (2002) Immunity 16: 157-168; Grant et al. (2002) J Exp Med 196: 1161-1171, Lee et al, 2006, (Nat Biotechnol. 2006 October; 24(10):1279-84) and Hishimoto et al 2010, J Exp Med. 2010 Jun. 7; 207(6):1135-43). Furthermore, treatment with a small molecule C5aR antagonist, a cyclic peptide can reduce inflammation and swelling after induction of arthritis in rats (Woodruff et al. (2002) Arthritis Rheum 46: 2476-2485).

Rheumatoid arthritis (RA) is an inflammatory disease of the joints. The affected joints are usually swollen and warm, and become increasingly more painful and stiff with time. Later symptoms include joint destruction including bone damage which severely affect the patient's movability and may include deformation of bones.

Although most drugs used for treatment of such inflammatory disease are mainly anti-inflammatory, it is of high importance and interest that a treatment of RA and related diseases also positively impact joint and bone destruction. In addition to RA, bone damage may occur in relation to other arthritis disease and even separate of an inflammation disease.

Osteoblasts and osteoclasts are collectively responsible for maintaining bones as osteoclasts may continuously break down osseous tissue while osteoblasts stimulate bone formation and thereby increase bone mass and/or bone density. Any compound that can influence the regulation of bone homeostasis and remodelling may thus be of relevance and have broad applicability in treatment of disease or disorders that affects the bones and in particular bone damage including bone erosion.

### SUMMARY

The inventors of the present application have found that C5aR antagonists have extended functionalities adding treatment of joint destruction, bone damage and in particular inhibition of bone erosion to the indications/symptoms where C5aR antagonists have usability.

One aspect of the invention relates to the use of C5aR antagonists for treatment or prevention of bone damage e.g. a C5aR antagonist for use in a method for treatment or prevention of bone damage. Bone damage is here considered to include bone erosion and loss of bone density. In addition the C5aR antagonists may further be useful for the combined treatment or prevention of bone damage and cartilage degradation. Such treatment or prevention may be for reducing the rate of bone damage and/or for improving or increasing bone mineral density. Bone damage is at least partly a symptom of inflammatory diseases affecting the joints, in particular arthritis and thus the C5aR antagonists may according to the invention be for use in treatment or prevention of bone damage in a subject suffering from one of the various forms of arthritis.

The inventors have surprisingly found that the effect of treatment using a C5aR antagonist provides a fast response as a therapeutic effect can be observed already after few dosages. In one embodiment the present invention relates to a C5aR antagonist for use in a method of treatment of prevention of bone damage, wherein an effect is obtainable after 4, 3, 2 or 1 dosage.

The functionality of the antagonists may according to the invention be considered linked to the ability of the antagonists to antagonize C5aR either by inhibiting or reducing binding of C5a to C5aR and/or by inhibiting or reducing C5aR mediated biological effect of C5a, such as a) C5a induced neutrophil activation, b) C5a induced cell migration and/or c) C5a induced neutrophil maturation.

A C5aR antagonist according to the invention may be any type of compound suited for therapeutic administration including antibodies and peptide molecules. A C5aR antagonist according to the invention would be administered as part of a pharmaceutical composition optionally including one or more pharmaceutical excipient. In one aspect the invention relates to a method for treatment or prevention of bone damage comprising administering a therapeutically effective amount of a C5aR antagonist as described herein, to an individual in need.

### SEQUENCE LISTING

SEQ ID NO.: 1: human C5aR
MDSFNYTTPD YGHYDDKDTL DLNTPVDKTS NTLRVPDILA
LVIFAVVFLV GVLGNALVVW VTAFEAKRTI NAIWFLNLAV
ADFLSCLALP ILFTSIVQHH HWPFGGAACS ILPSLILLNM
YASILLLATI SADRFLLVFK PIWCQNFRGA GLAWIACAVA
WGLALLLTIP SFLYRVVREE YFPPKVLCGV DYSHDKRRER
AVAIVRLVLG FLWPLLTLTI CYTFILLRTW SRRATRSTKT
LKVVVAVVAS FFIFWLPYQV TGIMMSFLEP SSPTFLLLKK
LDSLCVSFAY INCCINPIIY VVAGQGFQGR LRKSLPSLLR
NVLTEESVVR ESKSFTRSTV DTMAQKTQAV
2^{nd} loop is indicated by underlining of
AA 171-206.
SEQ ID NO. 2: mouse C5aR
MNSSFEINYD HYGTMDPNIP ADGIHLPKRQ PGDVAALIIY
SVVFLVGVPG NALVVWVTAF EPDGPSNAIW FLNLAVADLL
SCLAMPVLFT TVLNHNYWYF DATACIVLPS LILLNMYASI
LLLATISADR FLLVFKPIWC QKVRGTGLAW MACGVAWVLA
LLLTIPSFVY REAYKDFYSE HTVCGINYGG GSFPKEKAVA
ILRLMVGFVL PLLTLNICYT FLLLRTWSRK ATRSTKTLKV
VMAVVICFFI FWLPYQVTGV MIAWLPPSSP TLKRVEKLNS
LCVSLAYINC CVNPIIYVMA GQGFHGRLLR SLPSIIRNAL
SEDSVGRDSK TFTPSTDDTS PRKSQAV
2^{nd} loop is indicated by underlining of
AA 167-203.

### BRIEF DESCRIPTION OF DRAWINGS

FIG. 1 shows the binding of anti-C5aR mAbs to chimeric human/mouse C5aR. The figure indicates which regions of the C5a receptors proteins that are derived from human and mouse, as the mouse sequences are shown with a bold line and the human sequences are depicted with a thin line.

FIG. 2 shows C5a-induced CD11b expression quantified as A mean fluorescence intensity (FI): The mean Fl value for a given anti-C5aR mAb concentration is subtracted from the mean Fl value obtained from blood where no C5a is added. The result for mAb 20/70 is shown as a function of the mAb concentration. The dotted line represents the IC50 value, which was calculated to 0.6 μg/ml.

FIG. 3 shows that anti-C5aR reduces the histology score in the CIA mouse model. The graph shows the histology score index in mice treated with anti-C5aR, Enbrel® (Etanercept) and isotype control treatment, respectively. The histo-pathological end-point assessment of arthritic changes was performed on both hind paws from each mouse. Treatment with anti-C5aR significantly reduced the histology score index compared with the isotype control treatment.

FIG. 4 shows that anti-C5aR inhibits bone erosion and cartilage degradation in the CIA mouse model. Bone erosion and cartilage degradation was evaluated on HE stained sections of worst affected hind paw from each mouse. Treatment with anti-C5aR significantly reduces both bone erosion and cartilage degradation compared with mice treated with anti-TNP control antibody.

FIG. 5 shows that treatment with anti-C5aR reduces various arthritic parameters in a single paw arthritis model. Data was obtained from mice with DTH-arthritis treated with either blocking anti-C5aR antibody or isotype control antibody. Anti-C5aR treatment significantly reduces arthritic changes, including synovitis, cartilage degradation and bone erosion. In addition treatment significantly reduces extra-articular infiltration of inflammatory cells.

FIG. 6 shows that a single dose with anti-C5aR ameliorates arthritis 48 hours after treatment in the CIA mouse model. FIGS. 6A and 6B shows the mean clinical score (±SEM) per group over time and the AUC (area under the curve for individual clinical scores), respectively. The black line represents the mean. FIG. 6C shows the Histology score index. Both the clinical and histopathological assessment shows significantly reduced disease.

FIG. 7 shows that the disease activity in the DTHA model is reduced 60 hours following a single dose of anti-C5aR mAb. 7A shows the area under curve (AUC) of paw and ankle swelling in the two treatment groups measured over 60 hours after arthritis induction. Mean±SEM shown, n=10. *: p≦0.05; **: p≦0.01, Student's t-test. 7B shows a Semi-quantitative histopathology scoring of arthritic and inflammatory parameters in the two treatment groups on a scale of 0-3. Mice treated with anti-C5aR displayed lower degrees of synovitis, cartilage destruction, and bone erosion. n=10. *: p≦0.05; **: p≦0.01; ***: p≦0.001, Student's t-test with Welch's correction. 7C shows the sum of the individual scores in B (arthritis sum score), and sum of the individual scores in B minus extra articular infiltration (arthritis score). Maximum possible score is 15 and 9, respectively. n=10. *: p≦0.05; **: p≦0.01, Student's t-test with Welch's correction.

### DESCRIPTION

### Uses of C5aR Antagonists

Compounds or drugs that inhibit or reduce a biological response usually elicited by ligand-receptor interaction are termed receptor antagonists. Such receptor antagonist will bind the receptor but the interaction will not have efficacy. Presence of an antagonist will thus inhibit or reduce the biological effect of the ligand (or ligands) of the receptor. The action of antagonists may be medicated by binding an active site of the receptor thereby blocking or disrupting ligand interaction. Alternatively an antagonist may bind the receptor at a different site which also effective prevents ligand binding or receptor signalling.

C5aR antagonists have been found to be of relevance in treatment of inflammatory diseases and disorders based on results from several disease models of particular arthritis. The signalling mediated by the C5a receptor upon binding of C5a may thus be inhibited or reduced resulting an inhibition or reduction of the on-going inflammation process and thus relief of the swelling of joints. In addition it has been found that C5a and C5aR have a function in relating to bone homeostasis and remodelling. The inventors of the present application have further established that C5aR antagonists have favourable effects on bone damage.

An aspect of the present invention relates to a C5aR antagonist for treatment or prevention of bone damage or for use in a method for treatment or prevention of bone damage. In a similar aspect the invention relates use of a C5aR antagonist in the manufacture of a medicament for the treatment of bone damage.

The ability of C5aR antagonists to affect the development of bone damage may relate to one or more of bone erosion and loss of bone density. In general, treatment is a very broad term and covers multiple aspects including any effect that is seen as an advantage for a patient that is suffering from or at risk of developing bone damage, such as one or more of bone erosion and loss of bone density. In the present description treatment is used when referring to situations where bone damage has been observed whereas prevention is used when referring to situations where bone damage has not yet been observed. It is noted that in the examples of this document “treatment” refers to the mere administration of a compound irrespectively of any therapeutic effect by this administration.

In one embodiment the C5aR antagonist is for use in a method for treatment of bone damage (one or more of bone erosion and loss of bone density) and cartilage destruction.

Bone damage occurs by a continuous process that may result in severe effects on joint mobility. Bone damage in a given patient may have begun before that patient (or the physician) is aware that he or she is at risk of developing bone damage. In other cases the C5aR antagonist may be useful for prevention of bone damage in a patient at risk of developing bone damage. In one embodiment the C5aR antagonist may be for use in a method for prophylactic treatment of bone damage.

In some embodiments the C5aR antagonist is for use in a treatment to reverse or reduce bone damage e.g. to regenerate bone material of the joint. In a further embodiment the C5aR antagonist is for use in a treatment to improve bone mineral density.

It may be rare that bone damage can be fully prevented or fully treated and the ambition of a C5aR antagonist treatment may more often be to delay or halt progression of bone damage or to prevent further evolvement of bone damage.

In further embodiments the C5aR antagonist is for preventing bone damage, for reducing bone damage and/or for inhibiting bone damage. In further embodiments the C5aR antagonist if for delaying or halting of bone damage progression. In a further embodiment the C5aR antagonist is for inhibiting progression of bone damage.

In a further embodiment the C5aR antagonist is for use in a method for treatment of bone damage in a mammalian subject and in particular a human subject. As is apparent from the above the present invention relates to any aspect of treatment of bone damage in a human subject, such as preventing, inhibiting, halting and reversing of bone damage. Likewise the invention concerns treatment of bone damage including one or more of bone erosion and loss of bone density in a human subject.

The present invention thus relates to a C5aR antagonist for use in a method for treatment of bone damage in subject, wherein said subject is an individual in need. An individual in need is thus an individual that may benefit from treatment of bone damage, such as a patient suffering from bone damage or at risk of developing bone damage, again including one or more of bone erosion and loss of bone density.

In further embodiments the subject of treatment may be a patient suffering from arthritis. In one embodiment the subject has osteoarthritis (degenerative joint disease). In further such embodiments the osteoarthritis may be due to a trauma to the joint, an infection of the joint, or age related. In one embodiment the subject has rheumatoid arthritis which may also include subjects that have Juvenile idiopathic arthritis (JIA), also known as juvenile rheumatoid arthritis (JRA). In one embodiment the subject has septic arthritis. In one embodiment the subject has psoriatic arthritis.

In a further embodiment the subject may have gout or podagra (if the area of attack includes the big toe). In a further embodiment the subject may have spondylo arthropathies (SpA), also known as Bechterew's disease or Marie-Strümpell disease, including ankylosing spondylitis (AS) and Psoriatic Arthritis (PsA).

The criteria for diagnosis of arthritis may differ over time and depend on local praxis and the time when a given patient is examined by a specialist capable of diagnosing arthritis and the different forms hereof. In one embodiment the subject has one or more symptoms of arthritis, such as one or more symptoms of rheumatoid arthritis.

In a further embodiment a C5aR antagonist is for use in a method for treatment of bone damage, such as inhibiting, halting and/or reversing of bone damage to obtain a fast response. In one embodiment the C5aR antagonist is for use in a method for treatment of bone damage for obtaining a fast response. In such an embodiment, a fast is response is considered present if it is detectable after a limited number of dosages, such as after at most 5 dosages, such as at most 3 dosages, or preferably after as few as two or even one dosage of the C5aR antagonist. Depending on the C5aR antagonist employed, different timing of dosing is expected and to be determined case by case. The time for considering if the C5aR may be used for obtaining a fast response may thus vary between drugs. In one embodiment the fast response may be detected or detectable before the 5^{th} dosage is to be applied, such as before the 4^{th}, 3^{rd} or 2^{nd} dosage is to be administered. In one embodiment the fast response may be detectable before a second dosage is to be administered. If a once weekly dosing is foreseen a response may thus be expected within a months from 1^{st} dose, or within 4 weeks of 1^{st} dose or within 3 weeks of first dose, or within 2 weeks of 1^{st} dosage or within 1 week of first dose.

The fast response may be detected by various means known to the skilled artisan, as considered relevant for the disease and effect sought.

As the treatment of bone damage is not easily measurable in patients, alternative indirect means may be used.

The response may thus be detected by measurement of relevant disease biomarkers, such as markers describing the disease activity, such as cytokines and chemoattractants. Markers addressing the presence of specific cell types, such as MPO detecting neutrophils, as well as markers of tissue remodelling and/or extracellular matrix homeostasis may be used to evaluate if an early response is obtained. For clarity is noted that the fast response may be considered obtained also in situations where the effect is not actually measured e.g. situations where a response would have been detected if the relevant measurement had been performed.

In one embodiment a fast response is detected (or detectable) by measuring relevant markers locally or in the periphery, where peripheral signals are preferably detectable in a serum sample.

In one embodiment the C5aR antagonist is for use in a method of treatment of bone damage or bone erosion seeking a fast response as describe above wherein a relevant change of a neutrophils marker, as cytokine marker, a chemoattractant markers or a tissue remodelling marker can be observed. Examples of suitable markers may be found in table 1 and obviously further markers may be available to the skilled person.

In a further embodiment the C5aR antagonist is for oral administration. In a further embodiment the C5aR antagonist is for intravenous administration. In a further embodiment the C5aR antagonist is for subcutaneous administration.

In a further embodiment the C5aR antagonist is for daily administration. In a further embodiment the C5aR antagonist is for bi-weekly administration. In a further embodiment the C5aR antagonist is for once weekly administration. In a further embodiment the C5aR antagonist is for administration every second week. In a further embodiment the C5aR antagonist is administration with a frequency of every 20^{th}-25^{th} day, or such as once monthly.

### Methods of Treatment

An aspect the invention relates to a method for treatment or prevention of bone damage comprising administering a therapeutically effective amount of a C5aR antagonist to an individual in need. Within the scope of the invention, the method may cover variation and embodiments equal to the aspect and embodiments described above related to the uses of C5aR antagonists. Examples hereof are embodiments for treatment of a disease or disorder where delaying, halting and/or inhibition of bone damage progression is beneficial to the individual comprising administering a therapeutically effective amount of a C5aR antagonist to said individual. In one embodiment the method for treatment of bone damage comprising administering a therapeutically effective amount of a C5aR antagonist to an individual in need, wherein said C5aR antagonist is administered orally. In a further embodiment the C5aR antagonist is administered intravenously. In a further embodiment the C5aR antagonist is administered subcutaneously. In further embodiments the C5aR antagonist is administered once a day. In a further embodiment the C5aR antagonist is administered twice a week. In a further embodiment the C5aR antagonist is administered once a week. In a further embodiment the C5aR antagonist is administered every second week. In a further embodiment the C5aR antagonist is administered with a frequency of every 20^{th}-25^{th} day, or such as once monthly. In a further embodiment the C5aR antagonist is comprised by a pharmaceutical composition as described herein below.

In one embodiment the invention relates to a method for treatment of bone damage or bone erosion comprising administering a therapeutically effective amount of a C5aR antagonist to an individual in need, wherein a fast response is detectable. As described herein above a fast response may refer to a situation where a relevant change of a neutrophils marker, as cytokine marker, a chemoattractant markers or a tissue remodelling marker can be observed after a limited number of dosage or a limited time from 1^{st} administration.

In such an embodiment, a fast response is considered present if it is detectable after a limited number of dosages, such as after at most 5 dosages, such as at most 3 dosages, or preferably after as few as two or even one dosage of the C5aR antagonist.

Depending on the C5aR antagonist employed, different timing of dosing is expected and to be determined case by case. The time for considering if the C5aR may be used for obtaining a fast response may thus vary between drugs. In one embodiment the fast response may be detected or detectable before the 5^{th} dosage is to be applied, such as before the 4^{th}, 3^{rd} or 2^{nd} dosage is to be administered. In one embodiment the fast response may be detectable before a second dosage is to be administered. If a once weekly dosing is foreseen a response may thus be expected within a months from 1^{St} dose, or within 4 weeks of 1^{st} dose or within 3 weeks of first dose, or within 2 weeks of 1^{st} dosage or within 1 week of first dose.

### C5aR Antagonists

C5a, the A fragment of complement factor 5, binds its receptor C5aR and stimulates the inflammatory response. Inhibition of the C5a response with C5aR antagonists reduces the acute inflammatory response mediated via C5a without affecting other complement components. Different types of C5aR antagonists have previously been described (see background section) including peptide molecules such as cyclic peptide and anti-C5a receptor antibodies.

The critical feature for a C5aR antagonist is the ability of the molecule to interact specifically (in a given species) with C5aR, and not with other similar receptors such as C5L2, which is also a receptor for C5a. According to the invention a C5aR antagonist interacts specifically with C5aR. This interaction leading to inhibition or reduction of C5a binding to C5aR.

The terms “binding”, “specifically binding” and “binding specificity” is use herein to describe the selectivity of a C5aR antagonist.

The functionality of an anti-C5aR antagonist is dependent on the ability of said antagonist to significantly inhibit or reduce binding of C5a to C5aR. In one embodiment the invention relates a C5aR antagonist capable of significantly inhibiting or reducing binding of C5a to C5aR. This may be determined by a displacement assay (SPA) as described in Example 2 herein, from which IC50 values can be determined. In one embodiment the IC50 is below 50 nM. In a further embodiment of the invention the C5aR antagonist displaces C5a in an SPA assay, with an IC50 below 50 nM, such as below 40 nM, such as below 30 nM, such as below 20 nM, such as below 10 nM, such as below 5 nM or even below 4 nM, or with and IC50 below 3 nM or even below 2.5 nM or 2.0 nM.

The term binding “affinity” is used to describe monovalent interactions (intrinsic activity). Binding affinity between two molecules, e.g. an antagonist and a receptor, through a monovalent interaction may be quantified by determination of the dissociation constant (K_{D}) by measurement of the kinetics of complex formation and dissociation, e.g. by surface plasmon resonance (SPR) method. The rate constants corresponding to the association and the dissociation of a monovalent complex are referred to as the association rate constant kₐ (or kₒₙ) and dissociation rate constant k_{d} (or k_{off}), respectively. K_{D} is related to kₐ and k_{d} through the equation K_{D}=k_{d}/kₐ. Furthermore, “affinity” relates to the strength of the binding between a single binding site of a molecule (e.g., an antagonist) and a ligand (here receptor). The affinity of a molecule X for a ligand Y is represented by the dissociation constant (K_{d}), which is the concentration of Y that is required to occupy the combining sites of half the X molecules present in a solution. A smaller K_{d} indicates a stronger or higher affinity interaction, and a lower concentration of ligand is needed to occupy the sites. Similarly, the specificity of an interaction may be assessed by determination and comparison of the K_{D} value for the interaction of interest, such as a specific interaction between an antagonist and a receptor, with the K_{D} value of an interaction not of interest.

The term “significantly” is used to describe that an effect is of biological relevance, such as at least 10 or 20% inhibition or such as at least 10 or 20% induction.

The affinity of a C5aR antagonist may alternatively be determined in a competition ligand binding assay performed using neutrophils. This functionality is referred to as affinity of the antagonist as measured in a competition assay, but could also be considered measurement of the avidity of the interaction. The ex-vivo assays measures the ability of C5aR antagonist to neutralize C5a mediated actions in an in-vitro setting. In one embodiment the C5aR antagonist has an affinity below 1.0 nM or 0.80 nM, such as below 0.50 nM or 0.35 nM, as measured by competition ligand binding assay on neutrophils.

A further functional characteristic of a C5aR antagonist is the ability to inhibit C5a-dependent migration of neutrophils. This functionality may be evaluated as described in Example 2 herein. Examples of antibodies displaying this functionality have been described in WO2012/168199. In one embodiment the invention thus relates to a C5aR antagonist, wherein said C5aR antagonist inhibits C5a induced cell migration (in vitro or in vivo).

In one embodiment the invention relates to a C5aR antagonist capable of significantly inhibiting migration of human neutrophils.

In one embodiment the C5aR antagonist significantly inhibits migration of neutrophils in vitro.

In one embodiment the antibody inhibits migration to less than 50%, less than 40%, less than 30%, less than 20%, or less than 10% compared to the level of migration observed in the presence of 10 nM C5a and no C5aR antagonist. In one such embodiment migration is measured after 30 minutes in the presence of 10 nM C5a and C5aR antagonist and compared to the level of migration observed after 30 minutes in the presence of 10 nM C5a and no C5aR antagonist. Alternatively the ability of a C5aR antagonist to inhibit neutrophil migration can be express using IC50 values based on the same set up. In one such embodiment the IC50 is below 2.5 μg/ml, such as below 2.5 μg/ml, such as below 1.5 μg/ml, such as below 1.2 μg/ml or even below 1.0 μg/ml.

A further method to determine in vitro the functionality of a C5aR antagonist is a calcium-flux assay, that measures the ability of an C5aR antagonist to inhibit C5a induced neutrophil activation ex vivo, likewise described in Example 2. In a further embodiment the invention relates to an C5aR antagonist with an IC50 as determined in a calcium-flux assay below 7.0 μg/ml, such as below 5.0 μg/ml, such as below 2.5 μg/ml.

Additional ex vivo assays can be used to determine the ability of an C5aR antagonist to inhibit or neutralize C5a induced neutrophil maturation based on secondary effects such as CD11b and CD62L expression. CD11b and CD62L are maturation markers of neutrophils as they are up and down-regulated, respectively, upon activation by C5a/C5aR interaction.

In one embodiment, the invention relates to an C5aR antagonist with a IC50 as determined in an CD11b up-regulation assay is below 3.5 μg/ml, such as 3.0 μg/ml, such as below 2.5 μg/ml, such as below 2.0 μg/ml or such as 1.5 μg/ml or even below 1.0 μg/ml.

Likewise, the effect of the C5aR antagonist in a CD62L down-regulation assay may be determined. In one embodiment, the invention relates to an C5aR antagonist with a IC50 as determined in a CD62L down-regulation assay is below 1.8 μg/ml, such as below 1.5 μg/ml, such as below 1.2 μg/ml or even below 1.0 μg/ml.

The skilled person will be aware of further criteria to determine if a given compound is a suitable C5aR antagonist and may thus choose an assay of preference within the scope of this invention.

Although C5a and C5aR molecules share similarity across species, a C5aR antagonist may be species specific, such as specific for the human complex or the mouse complex. In one embodiment the antagonist is a human C5aR antagonist. In one embodiment the antagonist is a mouse C5aR antagonist.

Different types of molecules may have C5aR antagonistic function. In one embodiment the C5aR antagonist is an antibody.

As used herein, the term “antibody” is used to describe whole antibodies and any antigen binding fragments (i.e., “antigen-binding portion”) or single chain molecules thereof which specifically bind its corresponding antigen, here C5aR. Examples of antigen-binding fragments include Fab, Fab′, F(ab)2, F(ab′)2, F(ab)S, Fv (typically the VL and VH domains of a single arm of an antibody), single-chain Fv (scFv), dsFv, Fd (typically the VH and CHI domain), and dAb (typically a VH domain) fragments; VH, VL, VhH, and V-NAR domains; monovalent molecules comprising a single VH and a single VL chain; minibodies, diabodies, triabodies, tetrabodies, and kappa bodies (see, e.g., Ill et al., Protein Eng 1997; 10:949-57); camel IgG; IgNAR; as well as one or more isolated CDRs or a functional paratope, where the isolated CDRs or antigen-binding residues or polypeptides can be associated or linked together so as to form a functional antibody fragment. Various types of antibody fragments have been described or reviewed in, e.g., Holliger and Hudson, Nat Biotechnol 2005; 2S:1126-1136; WO2005040219, and published U.S. Patent Applications 20050238646 and 20020161201. Methods for generating antibodies, whole antibodies or antigen binding fragments are known in the art. Mouse monoclonal antibodies are typically made by fusing myeloma cells with the spleen cells from a mouse that has been immunized with the desired antigen. Human monoclonal antibodies can be obtained from transgenic animals (e.g. mice or other suitable species) encoding human antibodies. Alternatively, recombinant monoclonal antibodies can be made involving technologies, referred to as repertoire cloning or phage display/yeast display. Recombinant antibody engineering involves the use of viruses or yeast to create antibodies, rather than mice. Recombinant technologies may also be used for generation of “humanized” antibodies, which herein refers to a human/non-human chimeric antibody that contains sequences, usually at least the minimal complementarity-determining regions (CDR sequences) derived from a non-human germ line immunoglobulin sequence. A humanized antibody is, thus, a human immunoglobulin (recipient antibody) in which residues from a hyper-variable region of the recipient are replaced by residues from a hypervariable region of a non-human species (donor antibody) such as from a mouse, rat, rabbit, or non-human primate, which have the desired specificity, affinity, and capacity.

In one embodiment the C5aR antagonist is an antibody binding the 2^{nd} loop of C5aR. In one embodiment the C5aR antagonist is an antibody binding the 2^{nd} loop (AA 175-206) of human C5aR. In on embodiment the C5aR antagonist is an antibody binding AA 179-186 (EEYFPPKV). In one embodiment the C5aR antagonist is an antibody binding the corresponding 2^{nd} loop of mouse C5aR.

In such embodiments the antibody may be a monoclonal antibody, such as an antibody with the CDR sequences or variable regions of one of the antibodies described in any one of WO 03/062278, WO/022390 and WO2012/168199. A C5aR antagonistic antibody may be described as isolated to indicate that an antibody that has been separated and/or recovered from another/other component(s) of its natural environment and/or purified from a mixture of components in its natural environment.

In one embodiment the C5aR antagonistic antibody is of the IgG isotype, such as IgG1, IgG2 or IgG4.

Antibodies, via the Fc domain, interact with various Fc receptors and it therefore relevant to consider if such interaction is favourable or not as described in WO2012/168199, such as an antibody with one or more Fc mutations selected from E233P, L234A or V234A or F234L or F234V, L235E or L235A, G236R or G236A, G237A, S239D, S254W, N297Q, L328R, A330S, P331S and I332E. In one embodiment the antibody Fc is an IgG1 including 234A, L235E, G237A, A330S and P331S mutations. The antibody may additionally include a D327Q mutation.

In one embodiment the antibody according to the invention does not significantly induce phagocytosis of neutrophils in vitro, meaning that the level of phagocytosis is not significantly above background as measured in the absence of an anti-C5aR antibody. In one embodiment the antibody does not give rise to any detectable induction of phagocytosis. The assay for evaluating the level of phagocytosis may be performed using human neutrophils as described in Example 4 of WO2012/168199.

In an alternative assays the ability of anti-hC5aR antibodies to mediate cell depletion e.g. to induce ADCC (antibody dependent cellular cytotoxicity) and CDC (complement dependent cytotoxicity) may be evaluated. The assays apply C5aR expressing cells as target cells and effector cells (monocyte-depleted PMBCs) or complement containing sera to elicit the response. The assay is described further in Example 4 of WO2012/168199.

For the purpose of this application antibodies and fragments hereof are considered non-peptide compounds, as the term is here use in relation to smaller molecules. In one embodiment the C5aR antagonist is a non-peptide compound.

In one embodiment the C5aR antagonist is a peptide molecule, such as a peptide derived from C5a or C5aR. In one embodiment the C5aR antagonist is a cyclic peptide, such 3D53 (AcPHe-Orn-Pro-DCha-Trp-Arg) cyclized through the side chain of Orn and the terminal carbosylate (Wong et all, (Drugs. 1999 July; 2(7):686-93) which have been shown to inhibit binding of C5a to whole PMNs and to inhibit PMN degranulation as well (Finch et all, J Med Chem. 1999 Jun. 3; 42(11):1965-74). The compound 3D53 is also known as PMX53. In addition, a further similar compound is PMX205 (Woodruff T M: FASEB J, Vol. 20 (9), 1407-1417, 2006).

In addition to PMX53 and PMX205 described above further C5aR antagonists that are useful according to the invention may include one or more of MP-435 (Mitsubishi Tanabe/J&J), CCX-168 (ChemoCentryx/GSK), MEDI7814 (Medlmmune), ARC1905 (Ophtotech), NOX-D19 (Noxxon Pharma), ADC-1004 (Alligator Biosciences) and NGD 2000-1 (Neurogen).

The therapeutically effective amount of a C5aR antagonist must be determined for each compound and will depend on multiple causes, such as the potency, the bioavailability and in vivo half-life. The dosage to be administered will thus vary from compound to compound. In one embodiment where the C5aR antagonist is an antibody, the antibody may be administered in doses from 0.010 mg/kg up to 6 mg/kg.

### Pharmaceutical Formulations

The present invention further includes pharmaceutical compositions and/or formulations, comprising a pharmaceutically acceptable carrier and a C5aR antagonist according to the invention.

The C5aR antagonist according to the invention may in an aspect of the invention be used in the preparation of a pharmaceutical composition. Such a pharmaceutical composition may be prepared based on general knowledge in the field such as in the Pharmacopeia or Remington.

In an embodiment the pharmaceutical composition according to the invention comprise an antibody as described herein in combination with a pharmaceutically acceptable carrier. The formulation may be in the form of a liquid formulation or a dry formulation that is reconstituted in water or an aqueous buffer composition prior to administration. The formulation may be in the form of an aqueous formulation. In one embodiment the formulation may be a dry formulation in the form of a tablet or capsule. In an embodiment the formulation is sterilized.

A pharmaceutical composition of antibodies according to the invention may comprise a salt and/or buffer, such as the compositions described in WO2011/104381.

In further embodiment the pharmaceutical composition of an antibody according to the invention may be suitable for multiple uses, such as the compositions described in WO2011/147921.

In a further embodiment the pharmaceutical compositions of the C5aR antagonist may be for intravenous administration. In a further embodiment the pharmaceutical compositions of the C5aR antagonist is for subcutaneous administration.

In a further embodiment the pharmaceutical compositions of the C5aR antagonist is for oral administration.

In a further embodiment the pharmaceutical compositions of the C5aR antagonist is for daily administration. In a further embodiment the pharmaceutical compositions of the C5aR antagonist is for bi-weekly administration. In a further embodiment the pharmaceutical compositions of the C5aR antagonist is for once weekly administration. In a further embodiment the pharmaceutical compositions of the C5aR antagonist is for administration every second week administration. In a further embodiment the pharmaceutical compositions of the C5aR antagonist is administration with a frequency of every 20^{th}-25^{th} day, or such as once monthly.

The invention may further be described by, but not limited to, the Embodiments described here below. The findings are also illustrated by the Examples presented herein.

### EMBODIMENTS

1. A C5aR antagonist for use in treatment or prevention of bone damage.
2. A C5aR antagonist for use in treatment or prevention of bone damage, wherein bone damage includes one or more of: bone erosion and loss of bone density.
3. A C5aR antagonist for use in treatment or prevention of bone damage and cartilage degradation.
4. The C5aR antagonist according to any of embodiments 1-3, wherein the treatment is for reducing the rate of bone damage and/or cartilage degradation.
5. The C5aR antagonist according to any of embodiments 1-3, wherein the treatment is for inhibiting or halting progression of bone erosion.
6. The C5aR antagonist according to any of embodiments 1-3, wherein the treatment is for increase bone density.
7. The C5aR antagonist according to any of embodiments 1-3, wherein the treatment is for preventing bone erosion.
8. The C5aR antagonist according to any of embodiments 1-7, wherein the C5aR antagonist is for use in treatment of a mammal such as a human subject.
9. The C5aR antagonist according to embodiment 8, wherein the subject suffers from bone erosion or decreased bone density.
10. The C5aR antagonist according to embodiment 8, wherein the subject has one or more symptoms of arthritis.
11. The C5aR antagonist according to embodiment 8, wherein the subject has arthritis. 12. The C5aR antagonist according to embodiment 11, wherein the subject has osteoarthritis.
13. The C5aR antagonist according to embodiment 11, wherein the subject has rheumatoid arthritis.
14. The C5aR antagonist according to embodiment 11, wherein the subject has psoriatic arthritis
15. The C5aR antagonist according to embodiment 11, wherein the subject has septic arthritis.
16. The C5aR antagonist according to any of the previous embodiments, wherein the C5aR antagonist inhibits or reduces binding of C5a to C5aR.
17. The C5aR antagonist according to any of the previous embodiments, wherein the C5aR antagonist displaces C5a in an SPA assay, with an IC50 below 50 nM.
18. The C5aR antagonist according to any of the previous embodiments, wherein the C5aR antagonists inhibits C5a signalling via C5aR.
19. The C5aR antagonist according to any of the previous embodiments, wherein the affinity of the C5aR antagonists as measured in a competition ligand binding assay on neutrophils is below 0.80 nM.
20. The C5aR antagonist according to any of the previous embodiments, wherein the C5aR antagonists neutralizes C5a induced neutrophil activation ex vivo with an IC50 as determined in a calcium-flux assay below 7.0 μg/m.
21. The C5aR antagonist according to any of the previous embodiments, wherein the C5aR antagonists inhibits C5a induced cell migration.
22. The C5aR antagonist according to any of the previous embodiments, wherein the C5aR antagonists significantly inhibits migration of neutrophils in vitro.
23. The C5aR antagonist according to any of the previous embodiments, wherein the C5aR antagonists reduces migration to less than 50%.
24. The C5aR antagonist according to any of the above claims, wherein the C5aR antagonist reduces migration with and IC50 below 2.5 μg/ml.
25. The C5aR antagonist according to any of the previous embodiments, wherein the C5aR antagonist inhibits C5a induced neutrophil maturation ex vivo.
26. The C5aR antagonist according to any of the previous embodiments, wherein the C5aR antagonist inhibits C5a induced neutrophil maturation ex vivo with;
   a. an IC50 as determined in a CD11b up-regulation assay below 3.5 μg/ml, such as below 2.5 μg/ml, such as below 1.5 μg/ml or even below 1.0 μg/m or
   b. an IC50 as determined in a CD62L down-regulation assay below 1.8 μg/ml, such as below 1.5 μg/ml, such as below 1.2 μg/ml or even below 1.0 μg/ml.
27. The C5aR antagonist according to any of the previous embodiments, wherein the C5aR antagonists is a mouse C5aR (mC5aR) or human C5aR (hC5aR) antagonist.
28. The C5aR antagonist according to any of the previous embodiments, wherein the C5aR antagonist is an antibody.
29. The C5aR antagonist according to any of the previous embodiments, wherein the C5aR antagonist is an antibody binding the 2^{nd} loop of C5aR.
30. The C5aR antagonist according to any of the previous embodiments wherein the C5aR antagonist is an antibody which do not significantly induce phagocytosis of neutrophils in vitro.
31. The C5aR antagonist according to any of the previous embodiments wherein the C5aR antagonist is an antibody which do not significantly induce ADCC in vitro.
32. The C5aR antagonist according to any of the previous embodiments wherein the C5aR antagonist is an antibody which do not significantly induce CDC in vitro.
33. The C5aR antagonist according to any of embodiments 1-27, wherein the C5aR antagonist is a peptide, such as a peptide derived from C5a or C5aR.
34. The C5aR antagonist according to any of embodiments 1-27, wherein the C5aR antagonist is a peptide molecule, such PMX53 or PMX205.
35. The C5aR antagonist according to any of the previous embodiments to obtain a fast response.
36. The C5aR antagonist according to any of the previous embodiments for use in treatment of bone damage or bone erosion for obtaining a response.
37. The C5aR antagonist according to any of the previous embodiments 35 and 36 for use in treatment of bone damage or bone erosion wherein a fast response is detectable after at most 3 dosages.
38. The C5aR antagonist according to any of the previous embodiments 35 and 36 for use in treatment of bone damage or bone erosion wherein a fast response is detectable in a serum sample after at most 3 dosages.
39. The C5aR antagonist according to any of the previous embodiments 35 and 36 wherein a relevant change of one or more biomarkers is detectable after at most 3 dosages.
40. The C5aR antagonist according to any of the previous embodiments 35-39 for use in treatment of bone damage or bone erosion wherein a relevant change of one or more biomarkers is detectable after at most 3 dosages.
41. The C5aR antagonist according to any of the previous embodiments 35-40 for use in treatment of bone damage or bone erosion wherein a relevant change of one or more biomarkers selected from tissue remodelling or chemoattractants markers is detectable.
42. A pharmaceutical composition comprising a C5aR antagonist according to any of the above embodiments.
43. The pharmaceutical composition according to embodiment 42 further comprising a pharmaceutically acceptable carrier.
44. The pharmaceutical composition according to embodiment 42, wherein the formulation is a liquid formulation.
45. The pharmaceutical composition according to embodiments 42, wherein the formulation is a dry formulation.
46. The pharmaceutical composition according to embodiments 42-45, wherein the formulation is for intravenous administration.
47. The pharmaceutical composition according to embodiments 42-45, wherein the formulation is for subcutaneous administration.
48. The pharmaceutical composition according to embodiments 42-45, wherein the formulation is for oral administration.
49. The pharmaceutical composition according to any one of embodiments 42-48, wherein the formulation is for daily administration.
50. The pharmaceutical composition according to any one of embodiments 42-48, wherein the formulation is for bi-weekly administration.
51. The pharmaceutical composition according to any one of embodiments 42-48, wherein the formulation is for once weekly administration.
52. The pharmaceutical composition according to any one of embodiments 42-48, wherein the formulation is for every second week administration.
53. The pharmaceutical composition according to any one of embodiments 42-48, wherein the formulation is for administration with a frequency of every 20^{th}-25^{th} day, or such as once monthly.
54. A method for treatment or prevention of bone damage comprising administering a therapeutically effective amount of a C5aR antagonist to an individual in need.
55. A method for delaying, halting or inhibiting of bone damage progression comprising administering a therapeutically effective amount of a C5aR antagonist to an individual in need.
56. A method for increasing bone density, comprising administering a therapeutically effective amount of a C5aR antagonist to an individual in need.
57. The method according to embodiments 54, 55 or 56, wherein said C5aR antagonist is defined as in any of embodiments 16-42.
58. The method according to embodiment 57, wherein said C5aR antagonist is administered orally.
59. The method according to embodiment 57, wherein said C5aR antagonist is administered intravenously.
60. The method according to embodiment 57, wherein said C5aR antagonist is administered subcutaneously.
61. The method according to any of embodiments 57-60, wherein said C5aR antagonist is administered once a day.
62. The method according to any of embodiments 57-60, wherein said C5aR antagonist is administered twice a week.
63. The method according to any of embodiments 57-60, wherein said C5aR antagonist is administered once a week.
64. The method according to any of embodiments 57-60, wherein said C5aR antagonist is administered is administered with a frequency of every 20^{th}-25^{th} day, or such as once monthly.
65. The method according to any of embodiments 57-60, wherein said individual in need is an individual that may benefit from treatment of bone damage, such as an individual suffering from bone damage or at risk of developing bone damage, wherein bone damage include one or more of bone erosion and loss of bone density.
66. The method according to any of embodiments 57-65, wherein said individual in need suffers from arthritis, such as osteoarthritis, rheumatoid arthritis, septic arthritis or psoriatic arthritis.
67. The method according to any of embodiments 57-66, wherein said individual in need has one or more symptoms of arthritis, such as one or more symptoms of rheumatoid arthritis.
68. The method according to any of embodiments 57-67, wherein a pharmaceutical composition according to any of the embodiments 42-54 is administered.
69. The method according to any of the embodiments 57-68, wherein the method is for obtaining a fast response.
70. The method according to any of the embodiments 57-68, wherein the method is for treatment of bone damage or bone erosion for obtaining a response after at most 3 dosages.
71. The method according to any of the embodiments 57-68, wherein the method is for treatment of bone damage or bone erosion wherein a fast response is detectable after at most 3 dosages.
72. The method according to any of the embodiments 57-68, wherein the method is for treatment of bone damage or bone erosion wherein a fast response is detectable in a serum sample after at most 3 dosages.
73. The method according to any of the embodiments 57-68, wherein the method is for treatment of bone damage or bone erosion wherein a relevant change of one or more biomarkers is detectable after at most 3 dosages.
74. The method according to any of the embodiments 57-68, wherein a relevant change of one or more biomarkers is detectable after at most 3 dosages. 75. The method according to any of the embodiments 57-68, wherein the method is for treatment of bone damage or bone erosion wherein a relevant change of one or more biomarkers selected from tissue remodelling or chemoattractants markers is detectable.

### EXAMPLES

### Example 1

### Binding Region of 20/70 (Mouse Anti-mC5aR) and 7F3 (Mouse Anti-hC5aR)

The binding regions of 7F3 and mAb 20/70 to human and mouse C5aR, respectively, were determined and described (Lee et al. (2006) Nat Biotech 24 (10) 1279-1284). Briefly, chimeric human/mouse C5a receptors were constructed using overlap-extension PCR. Synthetic oligonucleotide primers were used to amplify regions of the human or mouse C5aR gene. The required human and mouse gene segments were joined to form a complete coding sequence for C5aR plus a C-terminal tag of 10 amino acids from bovine rhodopsin. Each DNA sequence was inserted into the mammalian cell expression vector pcDNA3.1(+) (Invitrogen) downstream of the constitutive CMV promoter. Expression vectors were transfected into mouse L1.2 cells using Lipofectamine 2000 (Invitrogen) according to manufacturer's instructions. Approximately 1.6 μg supercoiled plasmid DNA was added to 8×10⁵ cells. Transfected L1.2 cells were grown in RPMI 1640 (Gibco) supplemented with 10% bovine calf serum (Hyclone).

One day after transfection ˜6×10⁴ cells were centrifuged at 1,500 rpm for 5 min, washed once with PBS and re-suspended in 100 ul PBS containing 2% (wt/vol) BSA and 0.1% (wt/vol) sodium azide (staining buffer) and purified 20/70 antibody (5 ug/ml). After 30 min at 4° C., cells were washed twice with staining buffer and re-suspended in 50 μl FITC-conjugated donkey anti-rat IgG (Jackson Laboratories) diluted 1:200 in staining buffer. After incubating for 20 min at 4° C. cells were washed twice with staining buffer and analysed on the FACSCalibur (Becton-Dickinson) to determine the level of surface expression.

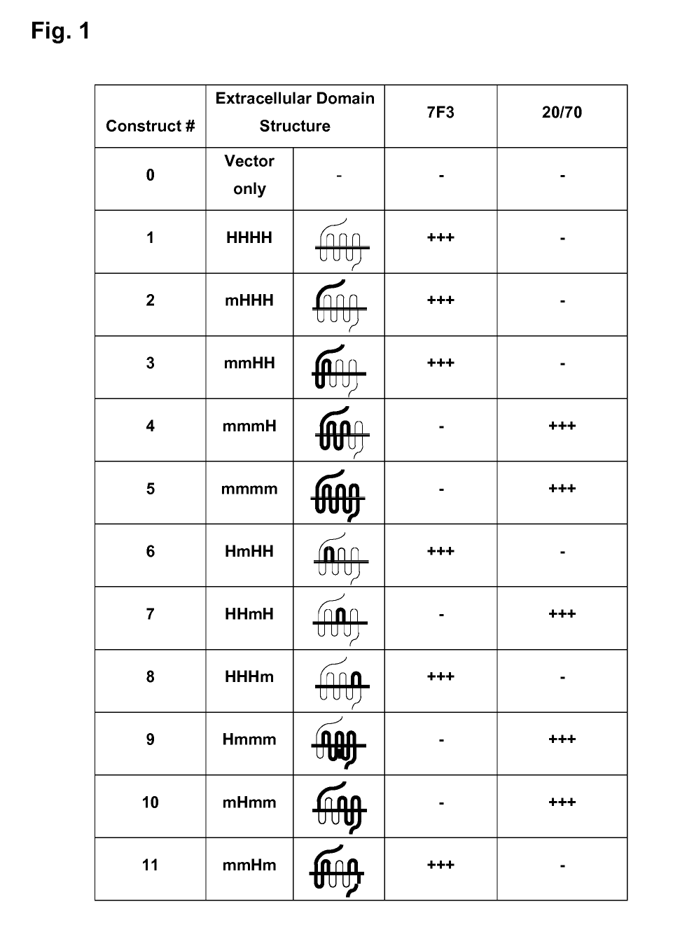

A series of chimeric receptors comprising segments of mouse and human C5aR were constructed to identify the region of the C5a receptor that antibody 20/70 bound. Each receptor construct contained none, 1, 2, 3 or all 4 of the mouse C5aR extracellular domains with the missing domains being the human C5aR sequence. The origin of the 4 extracellular domains (N-terminus, 1st, 2nd & 3rd extracellular loops) in each construct is shown schematically in FIG. 1, along with a summary of the FACS data.

FIG. 1 suggests that the anti-C5aR mAb 20/70 binds to the 2nd extracellular loop of mouse C5aR and that 7F3 binds to the corresponding 2nd loop in human C5aR. 20/70 binds to constructs 4, 5, 7, 9 and 10, all of which contain the mouse C5aR 2nd extracellular loop. Indeed, construct #7 comprises all human C5aR except for the 2nd loop which is mouse C5aR. Furthermore, 20/70 does not cross-react with human C5aR as evidenced by lack of binding to construct #1. Transfectants incubated in the presence of the FITC-conjugated secondary antibody only (i.e. no primary antibody) showed no staining (data not shown).

### Example 2

### In Vitro Tests of C5aR Antagonists

### CD11b Receptor Upregulation

Capacity of anti-C5aR mAb (m20/70) to inhibit up-regulation of CD11b expression in response to C5a.

### Assay Set-Up

The following set up was designed to determine the ability of C5aR antagonists to neutralize C5a-induced neutrophil maturation by measuring changes in CD11b expression.

### Materials and Methods:

Blood was collected into EDTA-coated tubes from naïve C57BL/6 mice (Taconic, Denmark) by puncture of the submandibular plexus. After collection the blood was pooled and 10 μl of anti-C5aR mAb (m20/70, Shushakova et al. 2002, Hycult biotech) in different concentrations (see table) was added to 11 aliquots of 90 μl pooled blood and incubated for 20 min. at room temperature (RT). After incubation, 9 μl 10 μg/ml recombinant mouse C5a (Hycult Biotech, HC1101) in PBS with 0.1% BSA (Miltenyi Biotech, 130-091-376) was added to each tube, except tube 1, and incubated for 20 min. at RT. Then each sample was stained for flow cytometry using 50 μl of a mastermix containing saturating concentrations of anti-Ly6G-PE (BD Biosciences, 55161) and anti-CD11b-AF700 (eBiosciences, 56-0112-82) in a staining buffer containing Cellwash (BD Biosciences, 349524) and 0.2% BSA (Miltenyi Biotech, 130-091-376). For staining of dead cells Fixable Near IR dead cell stain kit (Invitrogen, L10119) was added to the staining cocktail according to the manufacturer's instructions. Following incubation for 20 min. at 4° C. erythrocytes are lysed by adding FACS lysing solution (BD Biosciences, 349202) and incubating for 15 min. in the dark at RT. Cells are washed twice using staining buffer and subsequently analysed on a LSRII flow cytometer (BD Biosciences) using FACS Diva software.

| Tube | 1 | 2 | 3 | 4 | 5 | 6 | 7 | 8 | 9 | 10 | 11 |
|---|---|---|---|---|---|---|---|---|---|---|---|
| C5a (9 μl 10 μg/ml) | − | + | + | + | + | + | + | + | + | + | + |
| mAb μg/ml | 0.00 | 0.00 | 0.01 | 0.03 | 0.10 | 0.30 | 1.00 | 3.00 | 10 | 30 | 100 |

### Results:

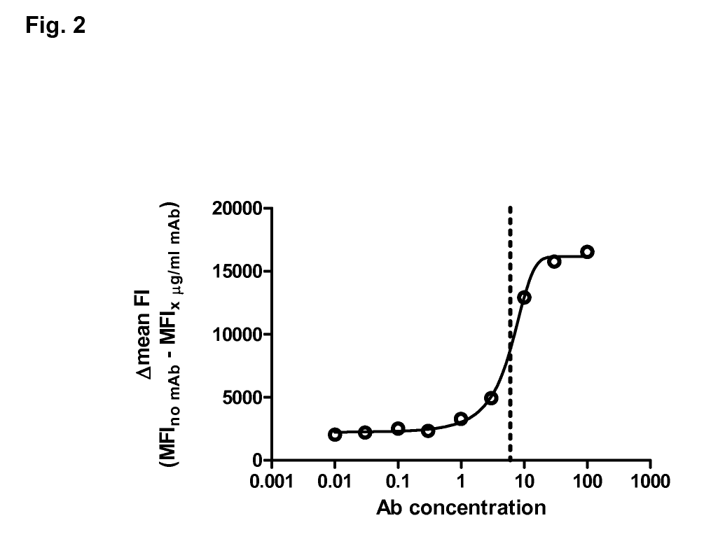

C5a-induced CD11b expression on neutrophils (defined as live Ly6G⁺ cells) was quantified as mean fluorescence intensity (FI) of AF700 for a given anti-C5aR mAb concentration subtracted from the value obtained from blood where no C5a was added (FIG. 2). The IC50 value for the anti-C5aR mAb was calculated as 0.6 μg/ml using non-linear curve fitting in GraphPad Prism software (v. 5.03).

It follows that the assay may be adapted to test other compounds evaluating there relevance as C5aR antagonists.

### CD62L Receptor Down-Regulation

### Assay Set-Up

The following set up is designed to determine the ability of C5aR antagonists to neutralize C5a-induced neutrophil activation by measuring changes in CD62L expression.

The above CD11b assay is adapted for CD62L detection by using a conjugated antibody recognizing CD62L (BD Biosciences, Cat. No 559772). The experimental details specific for CD62L are given below.

### FACS and Data Analysis

The FACSCalibur flow cytometer (BD Biosciences) is setup with compensation parameters established for channel FL-4. Samples are gated to exclude dead cells and debris. Neutrophils are identified as having high FSC and SSC and gated. The mean fluorescence intensity (MFI) of the gated neutrophils in the FL-4 (CD62L-APC) channel is calculated.

Results are expressed as a percentage of maximum CD62L expression with background subtracted. Maximum CD62L expression (MaxCD62L) is the average MFI of the neutrophils incubated without C5a and without C5aR antagonist. The minimum (background) CD62L expression (MinCD62L) is the average MFI of the neutrophils incubated with C5a but without C5aR antagonist. The formula used to calculate % of maximum CD62L expression for each samples was:

% MaXₛₐₘₚₗₑ=(MFIₛₐₘₚₗₑ−MFI_{Min})/(MFI_{Max}−MFI_{Min})×100

Data maybe entered into Graph Pad Prism (v4.0) and fitted to the sigmoidal dose-response curve (variable slope) i.e. 4-parameter logistic equation using non-linear regression to calculate the EC50.

### Displacement Assay

A Scintillation Proximity Assay (SPA) can be applied in order to determine the potency of an C5aR antagonist to displace C5a binding to C5aR. A detailed description of the SPA is provided in U.S. Pat. No. 4,568,649 and protocols provided by the manufacturer (Amersham Biosciences). Briefly, receptor-carrying membrane fragments purified from RBL-hC5aR cells bind to scintillating micro particles coated with wheat germ agglutinin (WGA). After addition of radio-labelled hC5a (¹²⁵I) tracer, binding to the receptors will result in emission of light from the particles. Specific for the SPA-principle, only radio isotope and particles in immediate proximity of each other will emit light. I.e. only radio-labelled hC5a bound to a receptor is close enough to a WGA-particle to produce light. The amount of light emitted is thus an expression of the amount of receptor-bound ¹²⁵I-hC5a. The assay is a competition assay, in which anti-hC5aR/unlabelled hC5a competes with the tracer on binding to the receptors. In the assay, a fixed amount of ¹²⁵I-labelled C5a is added to WGA-particles and C5aR receptors resulting in emission of a certain amount of light measured as counts per minute (cpm). If unlabelled C5a or anti-C5aR is added, binding hereof to the receptors will cause a lower cpm due to displacement of ¹²⁵I C5a. The % displacement is calculated as follows:

$$\frac{S - S max}{So - S max} \cdot 100 \%$$

S: Sample

Sₘₐₓ: Non specific binding. Measured by adding unlabelled hC5a in an amount sufficient to supersede the specifically bound ¹²⁵I-hC5a.

S₀: Maximum binding. No unlabelled hC5a is added.

The IC50 value is defined as the concentration which displaces 50% of C5a. The cpm is kept constant between experiments hence the IC50 values are relative as the tracer decades over time. The potency (IC50) of compounds to displace ¹²⁵I-hC5a may be used to identify C5aR antagonist.

### Neutrophil Migration (Chemotaxis) Assay

The potency of C5aR antagonists to inhibit hC5a (or mC5a) dependent neutrophil migration can be analysed in a Boyden chamber. Neutrophils isolated from human or animal blood are stained with calcein and added to the upper compartment in the Boyden chamber and mixed with the antibodies. hC5a or mC5a is applied to the lower compartment in the Boyden chamber and acting as chemoattractant for the neutrophils. The ability of neutrophils to migrate to the lower chamber is determined by counting the number of calcein-stained neutrophils passing through a 3 or 5 μm fluoroblok membrane.

Human PMNs (PolyMorphoNuclear leukocytes; granulocytes) are obtained from human blood samples drawn into vials containing EDTA. The blood cells areseparated by centrifugation of blood (4 parts) through a Ficoll-Paque PLUS (GE Health Care) gradient (3 parts) for 30 min (400×g) at room temperature. The PMN-containing layer is suspended in PBS (phosphate buffered saline) containing dextran-500 (Sigma) for 1 h to remove contaminating erythrocytes. The supernatant is centrifuged for 5 min (250×g) at room temperature and remaining erythrocytes are osmotically lysed using 0.2% NaCl for 55 s. The solution is made isotonic by 1.2% NaCl+PBS and centrifuged at 250×g for 5 min, before the osmotic lysis is repeated. After centrifugation the PMNs are resuspended in reaction mixture (RM): HBSS (cat no 14175 Gibco) contains NaCl 137 mM, KCl 5.3 mM, Na₂HPO₄ 0.33 mM, NaHCO₃ 4 mM, KH₂PO₄ 0.44 mM, Glucose 5 mM; supplemented with MgSO₄.7H₂O 0.4 mM, MgCl₂, 0.5 mM, CaCl₂ 0.5 mM, HEPES 20 mM. Cell density is determined by NucleoCounter (Chemometec). The PMN suspension should contain >95% neutrophils as evaluated by microscopy of Giemsa-stained samples.

Loading PMNs: Calcein, AM, (Fluka) is dissolved in DMSO (Dimethyl sulphoxide) and diluted 1000× in RM with cells (2×10⁶ cells per ml) to yield a concentration of 10 μM. The suspension is incubated for 30 min in incubator at 37° C. and then washed 3 times with RM to remove excess Calcein. Finally the cells are resuspended in RM (4×10⁶ cells/ml),

Migration is evaluated by the Boyden chamber technique using FluoroBlok® 3 μm pore size 96-well (cat. No. 351161.BD Falcon (VWR)). The upper chamber i.e. the inserts containing Fluoroblok membrane, is coated with human fibrinogen (cat no F3879-1 G, Sigma) in 1 mg/ml PBS at 37° C. for 2 hrs. After washing the membranes are blocked with a solution containing 2% bovine serum albumin (BSA), in PBS. After another wash using RM, 10⁵ Calcein-loaded PMNs with or without the C5aR antagonist are added to each well and placed in the receiver plate (lower chamber) which contained the control solution or the chemoattractant hC5a (Sigma, C5788). Each group comprised of at least 6 wells. Thereafter the plate is measured at 485/538 nm, 37° C. every 5 min for 60 min in a plate reader (SpectraMax, Molecular devices, or Fluoroscan, Thermo Labsystems.). The value at 30 min in relative fluorescence units is used as a measure of migration.

Curve fitting. The ability of C5aR antagonists to inhibit migration may be expressed by IC50 as determined using GraphPad Prism 5 (GraphPad Software, Inc.)

### Ex-Vivo Measurement of Affinity

The assay measures the ability of C5aR antagonist to neutralize C5a mediated actions in an in-vitro setting.

Isolation of Neutrophils from Fresh Human Blood

Blood is diluted in 1:1 with PBS+2% FBS and layered on Ficoll-Paque PLUS (GE Healthcare #17-1440-03) at a ratio of 3 parts Ficoll and 4 parts blood (15 ml Ficoll and 20 ml blood in a 50 ml tube) and subsequently stratified by centrifugation at 400×g for 30 minutes at RT. By aspiration the intermediate PBMC band is gently removed. The granulocytes stratified on the packed red cells are aspirated with a plastic Pasteur pipette. The granulocytes and red cells are transferred and pelleted in a new 50 ml tube. The pellet is diluted to 40 ml with 1x PBS and 10 ml of a 4% DEXTRAN 500 (sigma, 31392) solution in PBS (rate 1:5) is added and mixed gently by inversion. After 20-30 min. the granulocyte rich supernatant obtained is transferred to a new tube and spun down at 250×g for 5 min at RT. The contaminating red cells are removed with osmotic lysis by resuspending the cell pellet in 7.5 ml of 0.2% NaCl and gently mixing for 55-60 seconds. Subsequently 17.5 ml of 1.2% NaCl is added and then diluted to 50 ml with PBS and spun down at 250×g for 5 min. This step is repeated once. The cell pellets are subsequently resuspended in 1 ml reaction mixture (dPBS/RPMI). The viability and cell count is monitored using NucleoCounter®.

### Competition Ligand Binding Assay on Neutrophils

Human neutrophils are purified, washed and resuspended in binding buffer (50 mM HEPES, pH 7.5, 1 mM CaCl₂, 5 mM MgCl₂ and 0.5% bovine serum albumin (FractionV Ig G free)) at ˜5×10⁶ cells/ml. For each sample 40 μl cell suspension (1×10⁵ cells/well) is seeded into a 96-well V-shaped plate (Greiner, Cat. #651101). Competition studies are done using 12 concentrations of competing unlabeled ligand in half-log dilutions starting with 1 μM as the highest concentration. 40 μl of C5aR antagonist is added considering a final assay volume of 120 μl. 40 μl radioligand [¹²⁵I]-hC5a (Perkin Elmer, Cat. No. NEX250) is added to all samples except the background control. The final concentration of radioligand in the assay is 1 nM and the final volume is 120 μL. All samples are run in triplicate and incubated for 4 h at 4° C. Cells are then collected by centrifugation at 1200 rpm, at 4° C. for 2 min and washed three times in 100 μl of wash buffer (50 mM HEPES, pH 7.5, 1 mM CaCl₂, 5 mM MgCl₂, 150 mM NaCl and 0.5% bovine serum albumin (FractionV Ig G free)). Finally, cells are re-suspended in 30 μl wash buffer and transfered to an OptiPlate (Perkin Elmer, Cat. No. 6005290) and 150 μl of MicroScint 20 (Perkin Elmer, Cat. No. 6013621) is added to each well. The plates are covered, mixed well counted on a calibrated Top Counter with 1 h delay. The total amount of radioligand added to the assay is determined on a separate plate. The number of counts in each sample is expressed as normalized values in percentage where 100% is the maximum level of counts when 1 nM [¹²⁵I]-hC5a and no cold C5aR antagonist is added, and 0% is unspecific binding determined in the presence of 1 μM cold C5a. The data are analyzed by nonlinear regression using PRISM (GraphPad).

### Calcium-Flux Assay

Staining of Human Neutrophils with Fluo-4 AM Cell Dye

Neutrophils are centrifuged and washed in PBS then resuspended at 1×10⁷ cells/ml in Cell Dye and incubated at room temperature for 40 min in darkness. Cells are centrifuged and washed (to remove excess dye), centrifuged again and resuspended at 2×10⁶ cell/ml in Cell Buffer. Cells (0.5 ml) are aliquoted into non-sterile glass FACS tubes—one tube for each sample—stored at room temperature and used within two hours. Each sample used 1×10⁶ neutrophils.

### Assay

The calcium flux assay is carried out as follows. Briefly, 1×10⁶ neutrophils loaded with Fluo-4 AM in 0.5 ml Cell Buffer are analysed on a FACSCalibur flow cytometer (BD Biosciences) with neutrophils gated using x-axis FSC vs. y-axis SSC. The FL-1 (FITC) channel is used to measure neutrophil fluorescence following addition of various reagents to the tube (e.g. C5aR antagonist, C5a, ionomycin—dissolved at 10× final concentration in Cell Buffer rather than I-MGB or C-MGB). Sample fluorescence is measured continuously with a mean fluorescence intensity (MFI) value acquired every 1 second. This data is saved in a CellQuest (BD Biosciences) file and transferred to Excel (Microsoft) and Prism (v4.0c, GraphPad Software Inc.) for further processing and analysis. The order of adding reagents to the neutrophils and incubation times may be varied according to the type of assay carried out.

### C5a Neutralization Assay

A 10× 3-fold serial dilution of C5aR antagonist, with concentrations ranging from 1000 μg/ml to 1.37 μg/ml, is prepared. Fluo-4 AM loaded neutrophils (1×10⁶ in 0.5 ml Cell Buffer) are incubated with 50 μl 10× C5aR antagonist solution (final C5aR antagonist concentration in tube: 100—0.137 μg/ml) for 10 min at room temperature. Cells plus C5aR antagonist are analysed by FACS for ˜60 sec to establish baseline fluorescence. Then 50 μl 10 nM C5a is added to give a final concentration of −1 nM and fluorescence measurement continued for another ˜60 sec. If a C5aR antagonist blocks C5a-induced Ca²⁺-release then there is no spike of fluorescence. If the C5aR antagonist does not neutralize the C5a then there is a spike in fluorescence. Lastly, 50 μl 1 μg/ml ionomycin is added to a final concentration of 0.1 μg/ml and fluorescence measurement continued for another ˜60 sec to ensure cells are still responsive.

### Example 3

### Effects of Anti-C5aR in the Collagen Induced Arthritis (CIA) Model

### Mice

Male DBA/1 mice (10-11 weeks old) were obtained from Taconic (Denmark) and were allowed to rest for one week before initiation of the experiment. The mice were housed (10 mice/cage) under standard conditions and received standard diet and water.

Immunization for Induction of CIA Mice were immunized intradermally (i.d.) at the base of the tail with 100 μl bovine type II collagen (bCII, 2 mg/ml, Chondrex, USA) emulsified 1:1 in complete freunds adjuvans (2 mg/ml, Arthrogen-CIA®Adjuvant, Chondrex, USA) on day 0. On day 21 mice were given a boost immunization i.d. at the base of the tail with 100 μl bCII (1 mg/ml, Chondrex, USA) emulisified 1:1 with incomplete freunds adjuvans (Chondrex, USA). Mice were immunized under anaesthesia using isoflourane/O₂/N₂O.

### Clinical Scoring of Arthritis

Mice were scored from day 21 to the end of the experiment 5 times per week (all days except weekend days) according to the following scoring key: 0 points if no visible clinical symptoms

1 point/swollen toe (regardless of whether this includes one or two joints (—digit 1 in front paws), 1 point for involvement of knuckles, metatarsal/metacarpal, 1 point for involvement of wrist, tarsal/carpal. A maximum score of 6 could be obtained in front paws and a maximum score of 7 could be obtained in the hind paws. Mice that obtained a clinical score of more than 10 before experiment termination were sacrificed according to the definition of humane end-points by the local Danish authorities.

### Anti-Mouse C5aR mAb (M20/70)

The variable region for HC and LC was derived from the rat anti-mouse anti-C5aR mAb 20/70 (Shushakova et al. 2002, Hycult biotech). The constant region of LC is mouse kappa and constant region of HC is mouse IgG2a designed with 6 mutations in the FC region resulting in mlgG2a.1. The engineered mutations L234A, L235E, G237A (ADCC inactivation), D327Q, A330S and P331S (CDC inactivation) were made to reduce ADCC or CDC effector mechanisms. Lack of binding to mouse Fc-gamma receptors I-IV was determined by surface plasmon resonance analysis (data not shown).

### Test Compounds and Treatment of Mice

Individual therapeutic treatment of mice was initiated when mice obtained a score of 2-7 (most mice had score 2-4 at treatment initiation). Mice were randomized according to the treatment so that the first mouse qualifying for treatment initiation were given compound 1 and second mouse qualifying for treatment initiation were given compound 2 and so on. Using this procedure all compounds were represented in all cages and both early and late arthritis onset mice were represented in all dosing groups. All mice received either 6 doses over two weeks (with 1-2 days between treatments because no treatments were given during weekends). Mice were treated intraperitoneally (i.p.) with one of the following compounds:

20/70 anti-C5aR (mouse IgG2a.1) endotoxin level <0.10 EU/mg, 0.5 mg/mouse
Anti-TNP (mouse IgG2a.1) antibody, endotoxin level 0.001 EU/mg, 0.5 mg/mouse
Etanercept® 10 mg/kg
Anti-TNP (hzlgG1) antibody, endotoxin level <0.10EU/mg, 10 mg/kg

### Serum Samples

Mice were anaesthetized by isoflurane/O₂/N₂O and eye-bleed (app. 150 μl, max. 7.5% of estimated total blood volume) when treatment began and at termination. The blood was sampled into a 0.5 ml serum tube with clot activator (BD Microtainer) and were kept at room temperature until centrifugation (within 20 min.) at 15 000 G for 2 min. at 4° C. Serum was transferred to 1.4 ml micronic tubes and was stored at −80° C. until further analysis for matrix metalloproteinase-3 (MMP3) levels using a standard ELISA assay (R&D Systems). Serum samples were analysed using the assay procedure described by the matrix metalloprotinase-3 (MMP-3) ELISA manufacture R&D systems. Serum samples required a 1:20 dilution with Calibrator diluents. Additional inflammation markers were evaluated using a 58-biomarker multi-analyte profile (RodentMAP®, Myriad, RBM, Austin, USA).

### Preparation of Paws for Histology

At sacrifice paws where cut off and split sagitally between third and fourth toe to the heel bones in order to optimise fixation and decalcification. The paws were fixed in 4% paraformaldehyde for 48 hours at room temperature and placed in 70% ethanol. The paws were decalcified for 7 days in Immunocal and processed for paraffin embedding on an ASP300 tissue processor, and embedded with the cut surface down. Paraffin sections were cut at 3 μm and stained with a standard HE staining for histopathological evaluation.

### Histopathological Scoring System

Arthritic changes in the distal interphalangeal (DIP), proximal interphalangeal (PIP), metatarsal, and tarsal joints of the hind paws were assessed using a scoring system (modification of Sumariwalla et al, 2004) in which 0=normal joint architecture, 1=mild changes, synovitis and pannus front with few discrete cartilage focal erosions, 2=moderate changes, accompanying chondrocyte denucleation, loss of large areas of cartilage, eroding pannus front with some bone erosions, and synovial hyperplasia with infiltrating mononuclear cells and polymorphonuclear cells, and 3=severe bone erosions and destruction of joint architecture. The histology score index was calculated as the total additive score per mouse divided by the total number of joint areas scored per mouse (ranging from 6 to 8 joint areas per mouse).

For more specific scores of destructive changes in the paws, bone erosion and cartilage degradation were scored separately on the HE stained sections of the worst affected of the hind paws. Worst affected was defined as the paw with the highest histology score index (total additive score per paw divided by the total number of joint areas scored). If the two paws had the same histology score, the right paw was chosen. Severity of bone erosion and cartilage degradation was scored from 0-3, and the average was taken (additive score divided by the number of joint areas scored).

### Statistics

Statistical significant effects of test compounds on clinical scoring were calculated by comparing test compound groups with control groups using the two-tailed Mann-Whitney test. Calculations were done on individual mice evaluating the area under the curve (AUC) over the total experimental time for the clinical scores using Graph Pad Prism software. Mice that were sacrificed before end of experiment (after 6 treatment doses) were included in the calculation with the last observed score.

Histology data were analysed using Graph Pad Prism 5, and evaluation of statistical significance of the differences in the histology score index of the treatment groups was performed using unpaired Student's t-test with Welch's correction.

### Results

Therapeutic anti-C5aR treatment was able to halt disease development in the CIA model. A significant (p<0.0001) difference in the clinical scores were observed when comparing anti-C5aR treatment with isotype control treatment. In addition, a significantly (p=0.0002) reduced level of the systemic bone destruction marker MMP-3 confirmed the clinical treatment effect. Comparable inhibition of clinical scores and MMP3 level were found after etanercept treatment.

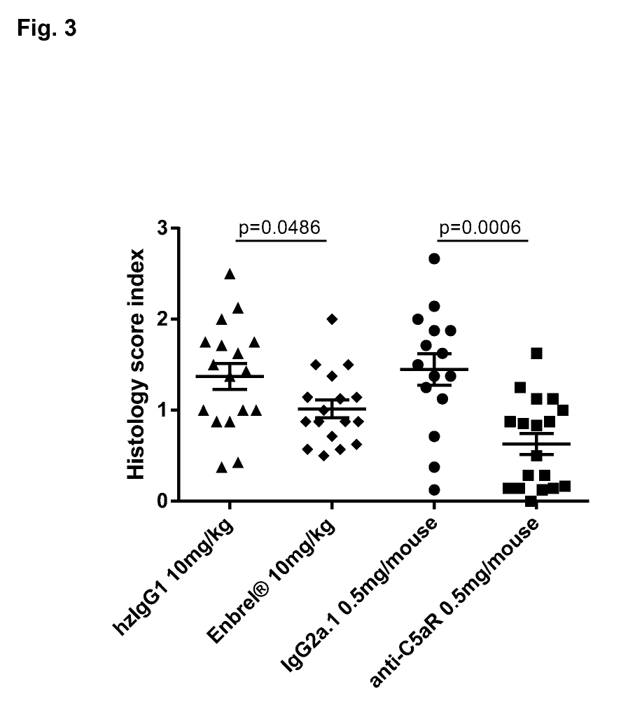

Histopathological end-point assessment of arthritic changes was performed on both hind paws from each mouse. A histology score index was calculated for each mouse. Treatment with anti-C5aR (n=18) significantly reduced the histology score index compared with the isotype control treatment (n=15) (FIG. 3). There was a significant correlation between the histology score index (the treatment groups were pooled) and the clinical score at the time of sacrifice (p<0.0001).

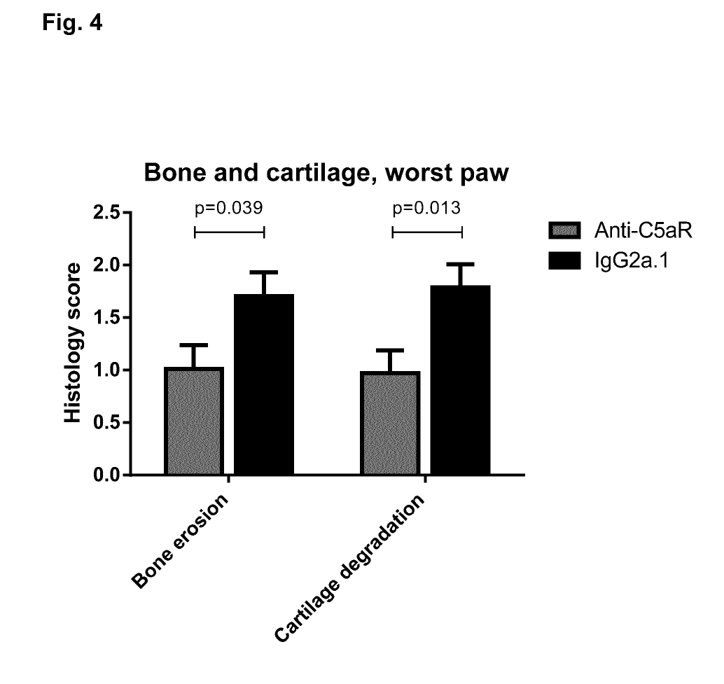

Bone erosion and cartilage degradation was evaluated on HE stained sections of worst affected hind paw from each mouse (anti-C5aR n=18, anti-TNP n=15). As seen in FIG. 4, treatment with anti-C5aR significantly reduces both bone erosion and cartilage degradation compared with mice treated with anti-TNP control antibody (IgG2a.1).

### Example 4

### Effect of Anti-C5aR Treatment in a Single Paw Arthritis Model in Mice

### Induction of DTH-Arthritis in C57BL/6 Mice

DTH-arthritis was induced by eliciting a classical DTH reaction in one paw with methylated bovine serum albumin (mBSA), with the modification that a cocktail of type II collagen monoclonal antibodies was administered between the immunisation and challenge steps (Atkinson S A et al. (2012) Arthritis Res Ther 14(3):R134). Briefly, female C57BL/6 mice (8-10 wk old) were immunised on day-7 with mBSA (Sigma, St. Louis, Mo.) emulsified in complete Freund's adjuvant (CFA) (Difco, Detroit, Mich.) intradermally at the base of the tail. Four days later they received 1000 μg (approx. 50 mg/kg) type II collagen (CII) mouse antibody 5-clone cocktail (Chondrex, Redmond, Wash.) containing the clones A2-10 (IgG2a), F10-21 (IgG2a), D8-6 (IgG2a), D1-2G (IgG2b), and D2-112 (IgG2b) intravenously in 200 μl phosphate-buffered saline (PBS). On day 0 the mice were challenged with 200 μg mBSA subcutaneously in 20 μl PBS in the right footpad. The left footpad was given 20 μl PBS only and served as control. After the footpad challenge mice develop a single paw arthritis in the right mBSA challenged footpad.

Treatment with Anti-C5aR Antibody

To investigate the effect of anti-C5aR treatment on the arthritis score, mice (n=10/group) were treated with either blocking anti-C5aR antibody (m20/70 mlgG2a.1 including 6 Fc mutations as described above) or isotype control antibody (anti-TNP-IgG2a.1), 500 μg i.p. twice weekly, 4 doses total from the day of arthritis induction (i.e. day 0).

### Quantative Measurement of Arthritis

As primary efficacy read-out the Area Under Curve (AUC) of Δ paw swelling was calculated from day 0-11 post arthritis induction, where the change (Δ) in paw swelling was calculated as: Right paw thickness post arthritis induction minus right paw thickness pre arthritis induction.

### Preparation of Paws for Histology

At sacrifice on day 11 post arthritis induction hind paws where cut off and split sagitally between third and fourth toe to the heel bones in order to optimise fixation and decalcification. The paws were fixed in 4% paraformaldehyde for 48 hours at room temperature and placed in 70% ethanol. The paws were decalcified for 7 days in Immunocal and processed for paraffin embedding on an ASP300 tissue processor, and embedded with the cut surface down. Paraffin sections were cut at 3 μm

### Assessment of Histopathological Changes

Histopathological changes in the paws were assessed on three sections from each paw stained with a conventional haematoxylin and eosin (HE) overview staining, osteoclast enzyme tartrate-resistant acid phosphatase (TRAP) staining for evaluation of bone erosion and Safranin O for demonstration of cartilage proteoglycan loss, respectively. Extra-articular infiltration of inflammatory cells (assessed on a 0-3 scale) and arthritic changes were assessed separately. Arthritic changes were assessed on metatarsal and tarsal joints, where synovitis (infiltration of inflammatory cells and hyperplasia of synovial membrane), cartilage degradation and bone erosion were scored separately on a 0-3 scale. For each of the three parameters of arthritic changes, an average between the two joint areas was calculated. Histological evaluation was performed on n=10 anti-C5aR treated mice and n=10 control antibody treated mice.

### Statistics

For statistical analysis of the histological scores an unpaired Student's t-test with Welch's correction was used. Differences between groups were considered significant when p≦0.05 and levels of significance were assigned as *: p≦0.05, **: p≦0.01 and ***: p≦0.001.

### Results

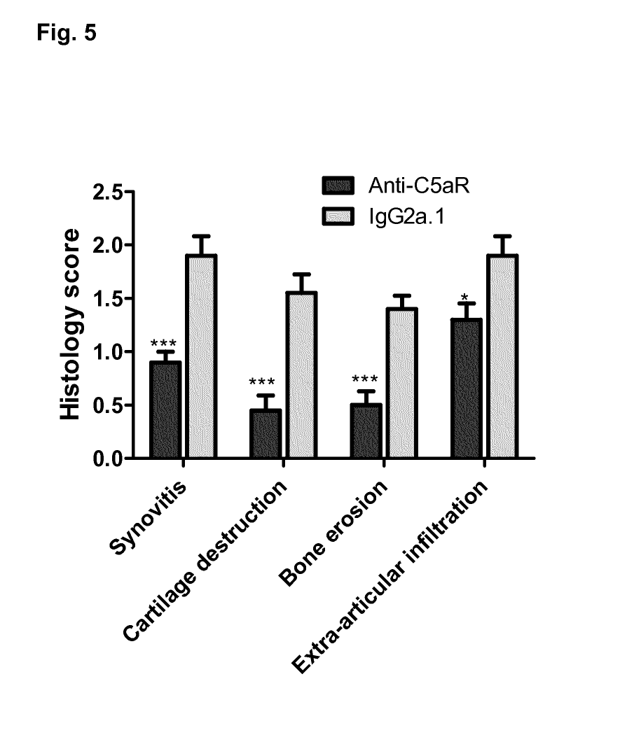

Anti-C5aR antibody treatment from the day of arthritis induction was shown to significantly reduce paw swelling compared to isotype control antibody (P<0.01, unpaired two-sided student's t-test, data not shown). The histopathological evaluation showed a highly significant effect of anti-C5aR treatment on arthritic changes (synovitis, cartilage degradation and bone erosion) and on extra-articular infiltration (FIG. 5). Together these findings show that anti-C5aR treatment can prevent arthritic changes in joints, which demonstrates the suitability of anti-C5aR as a therapeutic for the treatment rheumatoid arthritis (RA) and in addition for treatment of bone damage including bone erosion and loss of bone density.

While certain features of the invention have been illustrated and described herein, many modifications, substitutions, changes, and equivalents will now occur to those of ordinary skill in the art. It is, therefore, to be understood that the appended claims are intended to cover all such modifications and changes that fall within the true spirit of the invention.

### Example 5

### Single Dose Treatment of Anti-C5aR in the Collagen Induced Arthritis (CIA) Model

To study potential rapid onset effects of anti-C5aR treatment a single dose therapeutic treatment was evaluated. Mice were treated and analysed as described above (example 3). In short mice (n=22-26/group A-C) or (n=15-16/group D) were immunized for induction of arthritis at day 0 and boosted at day 21. Single dose treatment (0.5 mg/mouse) with anti-C5aR or anti-TNP (IgG2a.1) were initiated individually when mice reached a clinical score of 2-7 corresponding to score day 1. Mice were terminated 48 hours after the treatment. Inflammation markers were evaluated in paw homogenate using a 58-biomarker multi-analyte profile (RodentMAP®, Myriad, RBM, Austin, USA). The paw homogenate was prepared by cutting off the hind paws from all mice right below the fur line on the leg. The paws were then kept cold at all times and homogenised in a buffer containing: 1 tablet Complete (Roche), 5 μl Triton X-100 (Sigma), and 500 μl FUT-175 (Calbiochem) in 0.9 Saline (up to 50 ml). The homogenate was centrifuged two times for 15 min. at 10,000G at 4° C. and the supernatant was stored at −80° C. until analysis

### Results

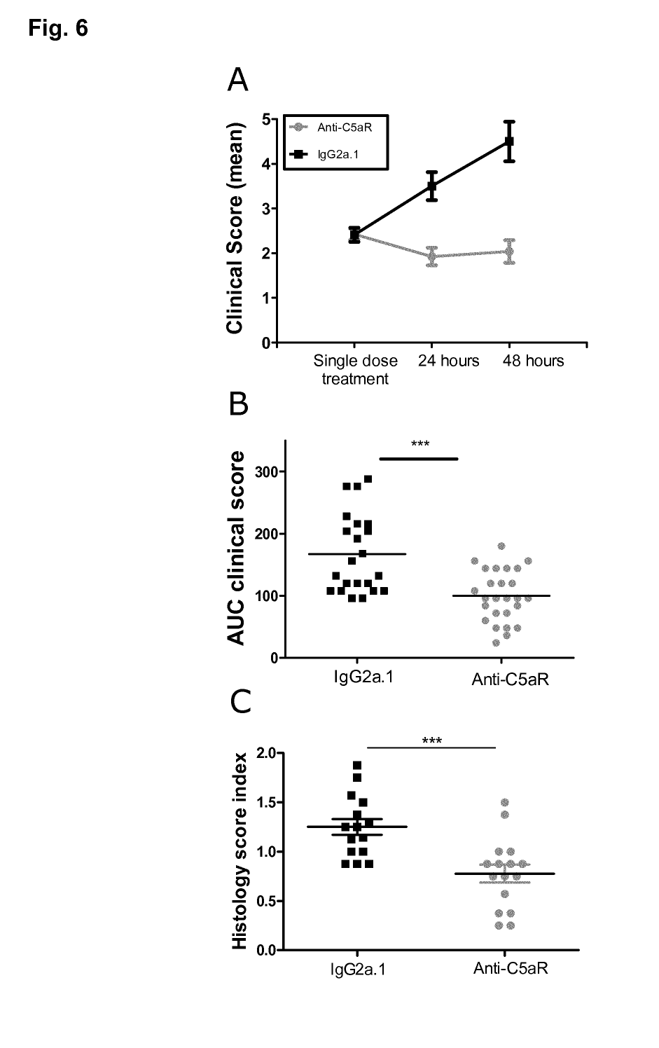

A single dose treatment with anti-C5aR demonstrated a halt in disease development already after 24 hours which was even more pronounced after 48 hours (FIG. 6A). The AUC of the clinical score was significantly lower (p=0.0003) in mice given single dose anti-C5aR treatment (FIG. 6B).

In a further study, the histopathology and infiltration of neutrophils and macrophages was also evaluated at 48 hours after single dose treatment. As above a significant reduction in the histology score index (p=0.0006) was observed (FIG. 6C). In addition the infiltration of neutrophils and macrophages were found to be significantly reduced at 48 hours (not shown). Together these data demonstrated a marked impact of single dose treatment with rapid onset effect on histopathology scores.

Single Dose Treatment with Anti-C5aR Results in Rapid Changes of Inflammatory Biomarkers

A panel of soluble analytes relevant to inflammation was investigated in paw homogenates and serum samples obtained from single dose treated mice 48 hours after administration of anti-C5aR. Many inflammation markers were found to be significantly reduced in mice that received anti-C5aR treatment compared to isotype (anti-TNP-IgG2a.1) treated mice (table 1).

In addition to a local response in the paw several inflammatory analytes were shown to be significantly decreased in the periphery after anti-C5aR treatment (table 1).

The matrix metalloprotinases MMP-3 and MMP-9 were both significantly (p=0.0028 and p=0.0006, respectively) reduced in the periphery corresponding to 35-45% reduction. In addition, a significant (p=0.0015) reduction of TIMP-1 corresponding to 47% reduction was found (table 1). This suggests that rapid onset effect of single dose anti-C5aR treatment on tissue remodeling and bone destruction can be detected in the periphery. As found in the paw homogenate, the chemoattractants KC (CXCL1), MCP-1 (CCL2), and MCP-5 (CCL12) were also significantly (p=0.0078, p=0.0028, p=0.021, respectively) reduced (20-50% inhibition) after single dose anti-C5aR treatment (table 1).

TABLE 1
Changes in inflammation markers after single dose anti-C5aR treatment. Only
analytes resulting in significant changes in either paw homogenate or serum are shown.

| | | % reduction of mean* | |
|---|---|---|---|
| Analyte | | Paw homogenate | Serum |
| Neutrophil marker | MPO | 72 (p = 0.0089) | 6 (ns) |
| Cytokines | TNF-α | 21 (p = 0.04) | LLOQ |
| | IL-6 | 69 (p = 0.013) | 40 (ns) |
| | IL-17A | 55 (p = 0.0061) | LLOQ |
| | IL-12p70 | 28 (p = 0.0036) | LLOQ |
| | IL-3 | 52 (p = 0.0094) | LLOQ |
| | IL-11 | 51 (p = 0.01) | LLOQ |
| Chemoattractants | KC (CXCL1) | 69 (p = 0.0037) | 46 (p = 0.0078) |
| | MCP-1 (CCL2) | 63 (p = 0.021) | 24 (p = 0.0028) |
| | MCP-3 (CCL7) | 60 (p = 0.023) | 19 (ns) |
| | MCP-5 (CCL12) | 63 (p = 0.0073) | 21 (p = 0.021) |
| | MIP-1b (CCL4) | 63 (p = 0.02) | LLOQ |
| | M-CSF-1 | 30 (p = 0.0073) | 8 (ns) |
| | IP-10 (CXCL-10) | 25 (p = 0.01) | 19 (ns) |
| | GCP-2 | LLOQ | 42 (p = 0.05) |
| Tissue remodelling | MMP-3^{#} | Not analyzed | 35 (p = 0.0028) |
| | MMP-9 | LLOQ | 38 (p = 0.0006) |
| | TIMP-1 | 63 (p = 0.019) | 47 (p = 0.0015) |
| | LIF | 64 (ns) | LLOQ |
| T cell activation | CD40L | 10 (ns) | 30 (p = 0.0057) |
| Cell death | APO-1/CD95 | 64 (p = 0.03) | 6 (ns) |
| Acute phase reactant | CRP | 62 (p = 0.0024) | 18 (ns) |

*anti-C5aR treated group compared with anti-TNP treated group (t-test) 48 hours after single dose treatment.
^{#}analysed by ELISA.
LLOQ; lower limit of quantification,
ns; non-significant,
M-CSF-1; macrophage colony-stimulating factor,
GCP-2; granulocyte chemotactic protein-2;
CRP; C-reactive protein,
LIF; leukemia inhibitory factor.

### Example 6

### Single Dose Treatment of Anti-C5aR in a Delayed Type Hyper-Sensitivity Arthritis (DTHA) Model

To further study the potential rapid onset of C5aR antagonists the effect was further evaluated in the DTHA model describe above (example 4) in a single dosage study.

Single dose treatment (0.5 mg/mouse) with anti-mC5aR or anti-TNP (mlgG2a.1) were administered i.p. The experiment was terminated 60 hours after treatment onset.

### Results

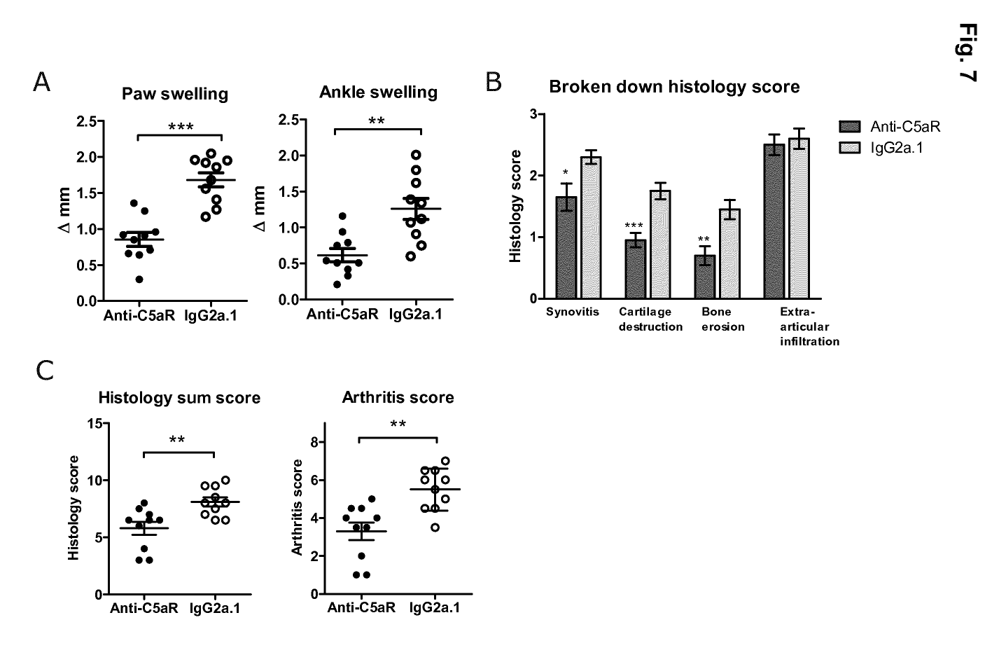

A single dose of anti-C5aR at time of arthritis induction led to a reduced paw and ankle swelling when measured 60 hrs later (FIG. 7A). Histopathological evaluation revealed that synovitis, cartilage destruction and bone erosion were attenuated, but extra-articular infiltration of inflammatory cells was not affected (FIG. 7B). The overall histopathology score was also reduced, as was the arthritis score, which is defined as the sum of all the individual evaluation parameters pertaining to an arthritic phenotype (FIG. 7C)

Together, these data demonstrate that a single dose of anti-C5aR had the ability to significantly reduce disease development and arthritic changes in DTHA

To further investigate the pathways affected by blockade of C5aR, and to investigate whether anti-C5aR lead to a reduction in activation of inflammatory cells, whole paw homogenates taken 60 hours post arthritis induction were analysed for protein levels of a range of cytokines and chemokines.

Briefly, paw homogenates were generated by homogenising paws in 1.25 ml of an ice cold homogenisation buffer containing 49.995 ml 0.9% saline, 1 tablet complete EDTA-free protease inhibitor cocktail (Roche, Switzerland), and 5 μl Triton X-100 (Sigma, St. Louis, Mo.). The paws were homogenised using a T25 Ultraturrax® homogeniser (IKA, Staufen, Germany) followed by centrifugation at 10,000 g for 15 minutes. The supernatants were decanted and centrifuged once more at 10,000 g for 15 minutes. The resulting supernatants were analysed. The homogenate supernatants were analysed undiluted for levels of inflammatory markers using bead-based Luminex® xMAP® technology with Milliplex kits from Millipore (Billerica, Mass., USA) according to the manufacturer's instructions. For statistical analysis, any values below the detection limit were set to the detection limit for the analyte in question and any values above detection limit was set to the upper detection limit for the analyte in question. In the case of VCAM-1, FGF (basic) and lymphotactin the analysis was performed using a 58-biomarker multi-analyte profile (RodentMAP®, Myriad RBM, Austin, USA).

The inflammatory cytokine IL-6 was significantly decreased in the paws of anti-C5aR-treated mice, while no change in IL-1β or IL-10 was observed. Furthermore, the chemoattractants CXCL1/KC, CXCL2/MIP-2, CXCL5/LIX and lymphotactin were significantly reduced in the paws of anti-C5aR-treated animals, as was G-CSF. Finally, vascular endothelial growth factor (VEGF), basic fibroblast growth factor (bFGF) and vascular cell adhesion molecule 1 (VCAM-1) were also significantly reduced in the paws of anti-C5aR-treated mice (table 2). In conclusion a single dose of anti-C5aR was able to mediate rapid changes in local inflammatory mediators, which are both markers of and important for the activation and infiltration of a variety inflammatory cell subsets and for endothelial activation.

TABLE 2
Protein biomarkers of inflammation in paw tissue following
a single dose of anti-C5aR or IgG2a.1

| | Anti-C5aR-treated, | IgG2a.1-treated, | p- | Summ- |
|---|---|---|---|---|
| Analyte | pg/g tissue (95% CI) | pg/g tissue (95% CI) | value | ary |
| IL-1β | 950.2 (765.6-1135) | 935 (774.6-1035) | 0.89 | ns |
| IL-6 | 1097 (582.3-1612) | 3873 (2235-5511) | 0.0028 | ** |
| IL-10 | 4403 (3418-5388) | 4280 (3320-5239) | 0.84 | ns |
| IL-17 | 47.98 (30.28-65.67) | 86.12 (43.34-128.9) | 0.066 | ns |
| CXCL1/ | 6854 (3919-9788) | 11630 (9374-13886) | 0.0092 | ** |
| KC | | | | |
| CXCL2/ | 7430 (4118-10742) | 20677 (13320-28035) | 0.0016 | ** |
| MIP-2 | | | | |
| CXCL5/ | 1134 (448.5-1819) | 3301 (2227-4376) | 0.0012 | ** |
| LIX | | | | |
| CXCL10/ | 4403 (3418-5388) | 4280 (3320-5239) | 0.84 | ns |
| IP-10 | | | | |
| CCL3/ | 3312 (2560-4065) | 3361 (2522-4200) | 0.92 | ns |
| MIP-1α | | | | |
| CCL5/ | 1015 (863.1-1167) | 1026 (835.1-1218) | 0.91 | ns |
| RANTES | | | | |
| G-CSF | 1681 (808.7-2562) | 6129 (2584-9675) | 0.013 | * |
| GM-CSF | 149.3 (57.3-241.4) | 260 (174.5-345.5) | 0.062 | ns |
| M-CSF | 1258 (957.7-1558) | 1040 (474.8-1604) | 0.45 | ns |
| VCAM-1 | 439 (371-506.9) | 503.4 (484-522) | 0.027 | * |
| VEGF | 74.49 (55.89-93.09) | 115.6 (89.77-141.5) | 0.0091 | ** |
| bFGF | 1567 (1048-2086) | 2349 (1705-2993) | 0.047 | * |
| Lympho- | 2351 (2003-2699) | 2836 (2416-3257) | 0.030 | * |
| tactin | | | | |

Whole paw homogenate supernatants were analysed for protein levels of a range of inflammatory markers using multiplex analysis. See materials and methods for details. Results are shown as mean±SEM, and n=10/group. Differences between groups were considered significant when p≦0.05 and levels of significance were assigned as *: p≦0.05, **: p≦0.01 and ***: p≦0.001 using Student's t-test.

1. A C5aR antagonist for use in treatment or prevention of bone damage of a subject having one or more symptoms of arthritis, wherein the C5aR antagonist is an antibody.

2. The C5aR antagonist according to claim 1, wherein bone damage includes one or more of: bone erosion and loss of bone density.

3. The C5aR antagonist according to claim 1, for use in treatment or prevention of bone damage and cartilage degradation.

4. The C5aR antagonist according to claim 2, wherein the treatment is for reducing the rate of bone damage and/or the rate of cartilage degradation.

5. The C5aR antagonist according to claim 1, wherein the treatment is for inhibiting or halting progression of bone erosion or for increasing bone density.

6. The C5aR antagonist according to claim 1, wherein the subject suffers from bone erosion or decreased bone density.

7. The C5aR antagonist according to claim 1, wherein the subject has arthritis, such as osteoarthritis, rheumatoid arthritis, psoriatic arthritis or septic arthritis.

8. The C5aR antagonist according to claim 1, wherein the subject is a mammal such as a human.

9. The C5aR antagonist according to claim 1, wherein the treatment is for obtaining a fast response.

10. The C5aR antagonist according to claim 1, wherein the C5aR antagonist inhibits or reduces binding of C5a to C5aR.

11. The C5aR antagonist according to claim 1, wherein the C5aR antagonist inhibits or reduces the C5aR mediated biological effect of C5a, such as
a. C5a induced neutrophil activation,
b. C5a induced cell migration and/or
c. C5a induced neutrophil maturation.

12. The C5aR antagonist according to claim 1, wherein the C5aR antagonist is an antibody binding the 2nd loop of C5aR.

13. A pharmaceutical composition comprising a C5aR antagonist according to claim 1.

14. A method for treatment or prevention of bone damage comprising administering a therapeutically effective amount of a C5aR antagonist to a patient in need thereof.

15. (canceled)


SEQUENCE LISTING (2 sequences)

SEQ ID NO: 1 - protein, 350 aa, Homo sapiens
  MISC_FEATURE (175)..(206): 2nd extracellular loop
MDSFNYTTPDYGHYDDKDTLDLNTPVDKTSNTLRVPDILALVIFAVVFLVGVLGNALVVWVTAFEAKRTINAIWFLNLAVADFLSCLALPILFTSIVQHHHWPFGGAACSILPSLILLNMYASILLLATISADRFLLVFKPIWCQNFRGAGLAWIACAVAWGLALLLTIPSFLYRVVREEYFPPKVLCGVDYSHDKRRERAVAIVRLVLGFLWPLLTLTICYTFILLRTWSRRATRSTKTLKVVVAVVASFFIFWLPYQVTGIMMSFLEPSSPTFLLLKKLDSLCVSFAYINCCINPIIYVVAGQGFQGRLRKSLPSLLRNVLTEESVVRESKSFTRSTVDTMAQKTQAV

SEQ ID NO: 2 - protein, 347 aa, Mus musculus
  MISC_FEATURE (171)..(196): 2nd extracellular loop
MNSSFEINYDHYGTMDPNIPADGIHLPKRQPGDVAALIIYSVVFLVGVPGNALVVWVTAFEPDGPSNAIWFLNLAVADLLSCLAMPVLFTTVLNHNYWYFDATACIVLPSLILLNMYASILLLATISADRFLLVFKPIWCQKVRGTGLAWMACGVAWVLALLLTIPSFVYREAYKDFYSEHTVCGINYGGGSFPKEKAVAILRLMVGFVLPLLTLNICYTFLLLRTWSRKATRSTKTLKVVMAVVICFFIFWLPYQVTGVMIAWLPPSSPTLKRVEKLNSLCVSLAYINCCVNPIIYVMAGQGFHGRLLRSLPSIIRNALSEDSVGRDSKTFTPSTDDTSPRKSQAV